(19) United States
(12) Patent Application Publication
(10) Pub. No.: US 2004/0029103 A1
(43) Pub. Date: Feb. 12, 2004

(54) Discrimination of cells using chemical characteristics

(76) Inventors: Mark R. Robinson, Albuquerque, NM (US); Keith Hutchinson, Corrales, NM (US); Shonn P. Hendee, Albuquerque, NM (US); Russell E. Abbink, Sandia Park, NM (US)

Correspondence Address: V. Gerald Grafe, esq., General Counsel, InLight Solutions, Inc., 800 Bradbury SE, Albuquerque, NM, 87106, US

(21) Appl. No.: 10/636,879

(22) Filed: Aug. 7, 2003

Related U.S. Application Data
(60) Provisional application No. 60/401,977, filed on Aug. 8, 2002.

(51) Int. Cl. C12Q 1/00

(52) U.S. Cl. 435/4

(57) ABSTRACT

The present invention allows detection of specific cell types based on chemical and functional characteristics of the cells. The invention can discriminate even between cells that are very similar; for example, the invention can discriminate between fetal and maternal red blood cells. The invention can also selectively alter certain cells; for example, by lysing cells of one type while leaving cells of another type unaltered. The invention has numerous applications. For example, the invention allows separation of fetal cells from maternal cells in maternal blood, allowing for fetal genetic screening without many of the drawbacks of current fetal cell acquisition techniques.

### CROSS REFERENCES TO CO-PENDING APPLICATIONS

This application claims priority under 35 U.S.C § 119 to U.S. provisional application No. 60/401,977, “Discrimination Of Cells Using Chemical Characteristics,” filed Aug. 8, 2002, incorporated herein by reference.

### FIELD OF THE INVENTION

The present invention generally relates to discrimination, including detection and selective modification, of cells based on their chemical characteristics. As a specific example, the present invention can be used to discriminate between fetal and maternal blood cells based on their spectroscopic response.

### BACKGROUND OF THE INVENTION

This invention describes a method for identifying and isolating fetal NRBCs from maternal cells in a maternal blood sample using intrinsic properties of the fetal NRBCs. This invention offers advantages over current techniques, which rely on nonspecific extrinsic biochemical labels to achieve fetal cell identification and isolation.

Prenatal diagnosis of fetal abnormalities is a common and important aspect of obstetric care. The goal of prenatal diagnosis is to accurately identify morphological, genetic, structural, and functional abnormalities of the fetus as early in pregnancy as possible. In the past, prenatal diagnosis was generally reserved for pregnancies in which there was an elevated risk of fetal abnormalities, such as advanced maternal age or family history of disease. Recent advancements in ultrasound and serological screening techniques have resulted in increased demand among patients and clinicians for prenatal screening and diagnosis (Paek et al. 2002. Prenatal Diagnosis. World Journal of Surgery 27:27-37).

A positive result in a prenatal screening test indicates an increased risk of fetal abnormality, at which point a prenatal diagnostic procedure may be indicated. Serological screening tests, which measure maternal serum levels of alpha-fetoprotein (AFP), human chorionic gonadotropin (hCG), and/or estriol, pose little or no risk to mother and fetus. Prenatal diagnostic techniques, on the other hand, require fetal genetic material, and the procedures for obtaining this material pose risk to the fetus. Current procedures for obtaining fetal genetic material, such as amniocentesis, chorionic villus sampling, and percutaneous umbilical blood sampling, are associated with increased risk of birth defects and miscarriage (Papp and Papp. 2003. Chorionic villus sampling and amniocentesis: Curr Opin in Obstet Gynecol 15:159-165). The risk of procedure-induced complications causes many couples to elect not to perform the diagnostic test. Though rare in occurrence, these complications associated with current prenatal diagnostic procedures have motivated clinicians and researchers to pursue less-risky diagnostic alternatives.

Recent research has been directed toward obtaining fetal genetic material from maternal blood samples. One approach being pursued by numerous researchers is to isolate one or more fetal nucleated red blood cells (NRBC) from a sample of maternal blood. Once isolated, NRBCs can be analyzed using standard laboratory techniques to diagnose trisomies, aneuploidies and genetic disorders. Researchers have shown that fetal NRBCs are present in the maternal blood, purportedly crossing from the fetal circulation into the maternal circulation by transfusion across the placenta. However, fetal NRBCs are present in the maternal circulation at extremely low concentrations. Researchers report that fetal NRBCs are present in maternal blood at concentrations of 1 to 500 fetal NRBCs per 20 ml maternal blood sample (1 to 500 NRBCs per 1010 maternal blood cells) (Wachtel et al. 2001. “Fetal Cells in Maternal Blood”, Clin Genet 59:74-79). The rarity of these cells in maternal blood poses a significant challenge for identifying and isolating them.

Current approaches for isolating fetal NRBCs typically involve first enriching the fetal NRBC concentration by subjecting the maternal sample to a gradient density separation step, which isolates mono-nucleated cells (maternal and fetal) from polynucleated and non-nucleated cells. The resulting sample of mononucleated cells is then tagged with fetal-cell specific biochemical markers to be used in either a magnetic-activated (MACS) or fluorescence-activated (FACS) cell-sorting step. These procedures are non-ideal for several reasons. Firstly, gradient density separation methods are imperfect, and some mononuclear cells are lost in this procedure. Secondly, the fluorescence and magnetic biochemical markers suffer from specificity shortfalls. Research conducted under the NIH Fetal Cell Study (NIFTY) has found MACS to have a 5% false-positive rate and FACS to have a 7% false-positive rate (Wachtel).

The prospect of isolating fetal NRBCs from a maternal sample holds promise as a less-risky alternative for prenatal diagnosis. However, the sensitivity and specificity of the method must be improved to make it clinically useful.

### SUMMARY OF THE INVENTION

The present invention allows detection of specific cell types based on chemical and functional characteristics of the cells. The invention can discriminate between cells that are very similar; for example, the invention can discriminate between fetal and maternal red blood cells. The invention can also selectively alter certain cells; for example, by lysing cells of one type while leaving cells of another type unaltered. The invention has numerous applications. For example, the invention allows separation of fetal cells from maternal cells in maternal blood, allowing for fetal genetic screening without many of the drawbacks of current fetal cell isolation techniques.

The invention encompasses various applications. In one, determining and examining the response to illumination of a sample allows detection of the presence of a selected type of cells. The environment surrounding the sample can be controlled to encourage a predictable response from the target cells. As a specific example, the oxygen pressure surrounding a sample containing maternal and fetal blood cells can be controlled. Fetal blood cells contain fetal hemoglobin, which has a greater oxygen affinity than does adult hemoglobin. Controlling the oxygen pressure can foster a condition where the fetal blood cells are oxygenated while the majority of maternal blood cells are not oxygenated. Response to illumination can be used to detect the presence of oxygenated hemoglobin, and consequently the presence of fetal blood cells, in the sample. For example, oxygenated hemoglobin can exhibit a spectroscopic response to illumination (e.g., visible or infrared light) different than that exhibited by non-oxygenated hemoglobin. Detecting the spectroscopic response of oxygenated hemoglobin from cells in an environment where only fetal blood cells will be oxygenated can indicate the presence of fetal blood cells.

As another application, cells of a specific type can be isolated from a sample. Cells can be detected using similar principles as described for the detection application. The detection can be localized on a per cell basis, for example by imaging the response, or by cell-by-cell analysis (using flow cytometry, for example). The cells exhibiting the response expected for the desired cell type can then be isolated using any of a variety of techniques: cell gating as in flow cytometry, individual cell selection as in laser tweezers (“The Micro-Robotic Laboratory: Optical Trapping and Scissing for the Biologist”, incorporated herein by reference), selective destruction (by destroying all but the identified cells, for example by destructive laser illumination, avoiding the identified cells).

As another application, cells of a specific type can be selectively altered. A sample can be exposed to radiation having an intensity vs. wavelength characteristic that is more strongly absorbed by one type of cells than by another. The incident radiation can be tailored to encourage changes in the strongly absorbing cells, for example by supplying sufficient energy at absorbed wavelengths to lyse the absorbing cells, either in bulk or on a cell-by-cell basis.

As another application, cells whose response characteristics vary with changing environmental conditions, for example the change in the hemoglobin absorption spectrum based on oxygenation status, can be used to detect those environmental conditions.

### BRIEF DESCRIPTION OF THE DRAWINGS

FIG. 1 is an illustration of hemoglobin spectra.

FIG. 2 is a representation of the shift in the oxygen dissociation curve seen in a newborn and during the first 11 months of development.

FIG. 3 is an illustration of the development life cycle of a red blood cell.

FIG. 4 is a schematic illustration of an apparatus according to the present invention.

FIG. 5 is a representation of an example distribution of absorbance signals obtained from a mixture of maternal and fetal cells containing various representative percentages of fetal hemoglobin (HbF).

### DETAILED DESCRIPTION OF THE INVENTION

Fetal Cell Separation Example Application

A detailed description of detection of fetal blood cells, and isolation of fetal blood cells from maternal bloods cells, using response to infrared radiation, will be presented. Those skilled in the art will appreciate other applications of the invention, using a similar detection method adapted for specific environmental conditions and cell properties. In the example application, the present invention detects fetal blood cells within a sample of maternal blood. Existing technology, such as FISH assays, can be used to perform genetic analysis of the fetal blood cells, for example for diagnosis of trisomy 13, 18, and 21, and aneuploidies of sex chromosomes. Such a test would likely be indicated for all pregnant women under the age of 35, and for all pregnant women over the age of 35 who did not undergo amniocentesis.

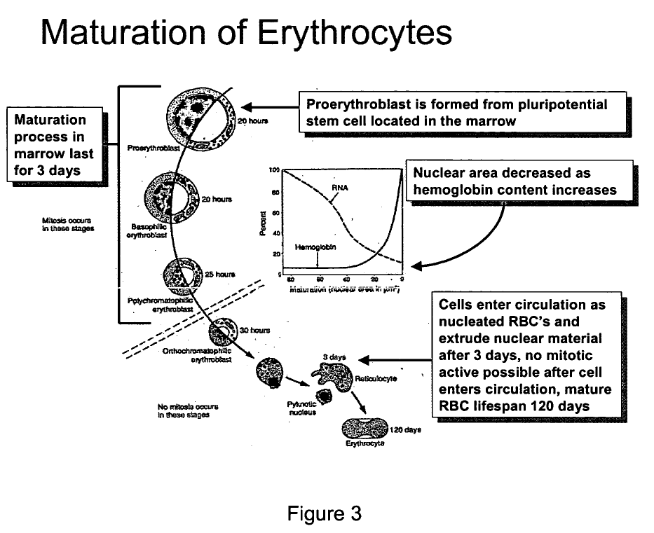

Fetal blood cells are produced as nucleated cells. Approximately three days after entering circulation, the cells exude the nuclear material, as illustrated in FIG. 3. Fetal blood cells can be found in maternal blood; for the short time before they exude their nuclear material they contain genetic information from the fetus.

Fetal hemoglobin (HbF) has a greater oxygen affinity than does adult hemoglobin (HbA, HbA2). Controlling the partial pressure of oxygen can therefore produce an environment where cells containing fetal hemoglobin are oxygenated while other blood cells are not. Since hemoglobin responds differently to radiation based on its oxygenation state, blood cells under controlled oxygen conditions can be identified as containing fetal hemoglobin or not based on their response to radiation. Fetal hemoglobin is present in greater concentration in fetal blood cells than in maternal blood cells; consequently, those cells identified as containing higher levels of fetal hemoglobin are more likely to be fetal blood cells.

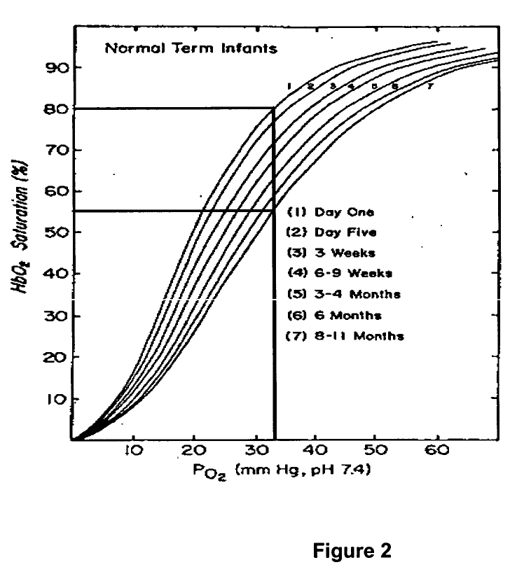

More specifically, fetal red blood cells contain a large proportion of hemoglobin F (Hb F). This Hb F concentration in the fetus can comprise from 50 to 85% of the all the hemoglobin found in a sample of fetal cord blood. The other portion of the hemoglobin in the sample is adult hemoglobin (Hb A and Hb A2). Most of this hemoglobin is Hb A as only trace amounts of Hb A2 are present at birth (0.3%). Maternal red blood cells are generally comprised of a ratio of Hb A and Hb A2. Maternal cells can contain Hb F under certain condition to include pregnancy. Hb F has an affinity for oxygen that is greater than that of Hb A or Hb A2. This allows the fetus to fully oxygenate its blood at a lower partial pressure of oxygen (pO₂) than is seen in the alveoli of the maternal lungs. FIG. 2 represents the shift in the oxygen dissociation curve seen in a newborn and during the first 11 months of development. For the purpose of this discussion consider the 11 month curve as representative that of the mother. This is a valid assumption due to the fact that by age 8-11 months the percentage of HbA in the baby approaches adult levels. For genetic testing, nucleated red blood cells are needed (FNRBC). These occur, on average, as one cell per 10⁹ to 10¹⁰ maternal red blood cells, or about 10 to 100 FNRBCs in a 20 ml sample of maternal blood.

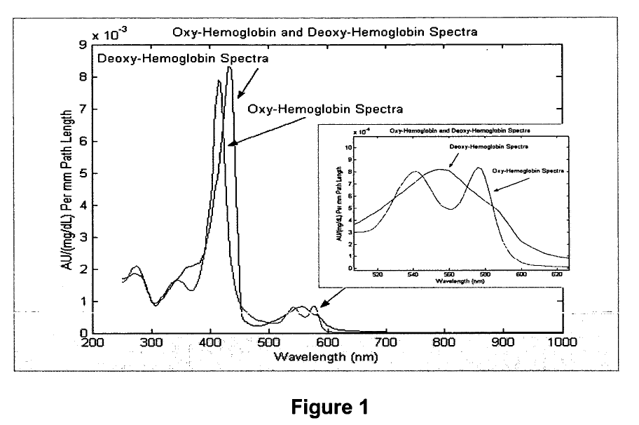

The difference in response between the two types of cells can be determined by analyzing the full absorbance spectrum of hemoglobin (partially illustrated in FIG. 1); it can also be determined by analyzing a subset of the spectrum sufficient to provide the desired discrimination. For example, two single wavelength signals can come from the hemoglobin molecule. The change in signal can be based on the different spectra seen between the oxygenated hemoglobin (oxy-hemoglobin) molecule and that of the deoxygenated (deoxy-hemoglobin) molecule. One area of interest is in the visible region between 400 and 600 nm. This area has been enlarged in FIG. 1.

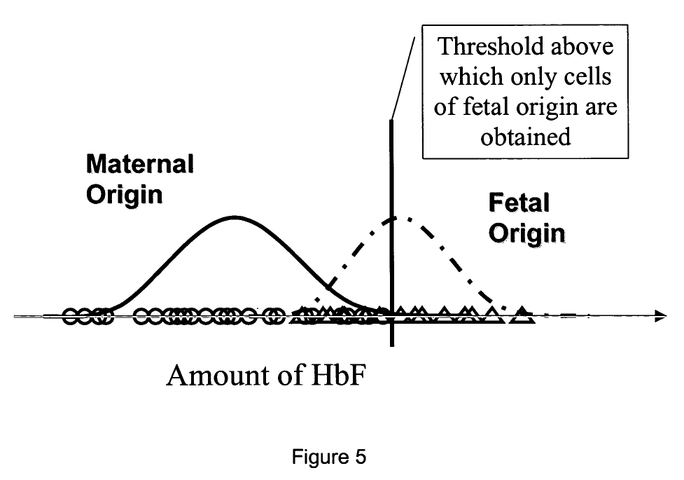

Due to different oxygen affinities, Hb F can be identified in the presence of Hb A and Hb A2. In a proposed application, cells containing Hb F can be selected based upon their response to a defined oxygen environment and their response to illumination. As noted above, Hb F is present in cells of fetal origin as well as cells of maternal origin. Based upon research conducted at Tufts University and other laboratories, it is recognized that the amount of Hb F in cells of fetal origin will exceed on average the amount of Hb F present in cells of maternal origin (Bohmer et al. Flow cytometric method for the detection of fetal nucleated red cells in cultures of maternal blood, in Macek et al., eds, “Early prenatal diagnosis, fetal cells and DNA in the mother. 12^{th} Fetal Cell Workshop, Prague, 2001. pp. 79-86). Beer's law states that the absorption of an illumination signal by a spectrally active chemical is proportional to the concentration of the chemical in the sample illuminated. Thus, maternal and fetal cells containing Hb F can be separated using a quantitative assessment of the absorbance signal generated. FIG. 5 shows an example of the distribution of absorbance signals obtained from a mixture of maternal and fetal cells containing Hb F as well as a threshold that enables the selection of those cells of fetal origin.

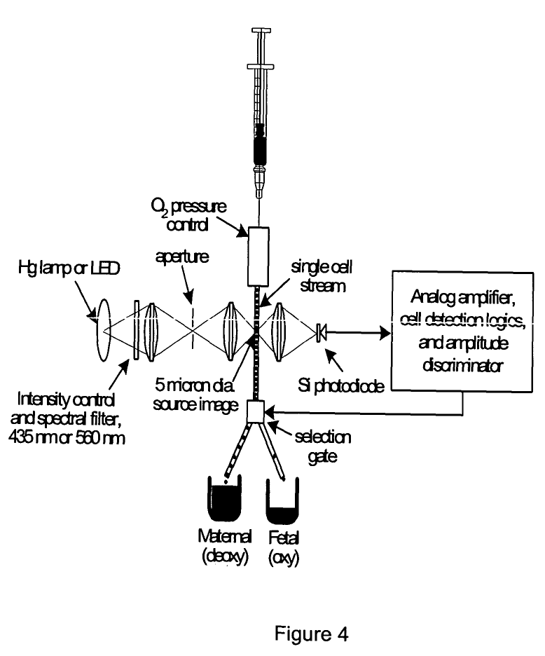

Flow cytometry technology can be used to create a stream of cells one cell thick. The response of each cell to incident radiation can be analyzed to determine whether the cell contains oxygenated hemoglobinor not, and therefore can determine if the blood cell is of fetal origin or not. The cells can then be gated to a waste container or a storage container based on their spectral signal. FIG. 4 shows an example of such an apparatus. Other cell separation techniques can also be used, for example parallel flow cytometry. Also, the cells can be deposited in substantially a monolayer, and the present invention used to identify specific cells of interest. Cell manipulation technologies such as laser tweezers can be used to select the desired cells. Also, conventional energy control technologies such as those used in laser printers can be used to alter or destroy either the fetal or maternal cells based on information from applying the present invention to the monolayer sample.

Centrifugation techniques can be used to separate nucleated cells from non-nucleated cells, enriching the population of nucleated fetal cells in a sample.

As an example of the performance attainable, consider the signal obtainable given one set of assumptions, using irradiation from a 50W Hg arc lamp with a spectral filter to select 436 nm line with 4 nm half amplitude width and a 1 mm diameter, and a Silicon photodiode detector, as shown in Table 2. The example signal to noise ratio would allow 100,000 cells to be sampled per second with a single detector.

TABLE 2

| | |
|---|---|
| Hemoglobin absorption coefficients in AU | 435 nm: Deoxy = 8.4 × 10⁻³; |
| (mg/dl)⁻¹ (mm of path length) | Oxy = 3 × 10⁻³; delta = |
| | 5.4 × 10⁻³. |
| Minimum cell diameter: | 1.0 microns = path length |
| Normal hemoglobin concentration | 14 g/dl |
| Difference in HbO2 saturation at | 25% = fraction of delta |
| 33 mm Hg | realizable |
| Irradiation | |
| Arc size | 0.2 × 0.35 mm |
| | 40 × source image reduction |
| | to produce 5 micron diameter, |
| | NA 0.7 spot on cell |
| estimated power on cell | 4 × 10⁻⁵ W |
| Detector | |
| NEP | 1.6 × 10⁻¹⁵ W/Hz^{1/2} |
| responsivity at 436 nm | 0.2 NW |
| Shot noise limited NEP | 9 × 10⁻¹² W/Hz^{1/2} |
| Shot noise limited NEP at | 5.7 × 10⁻⁹ W |
| 10⁵ measurements | |
| per second | |
| Expected signal at 435 nm | 5.4 × 10⁻³ × 14,000 × 0.001 × |
| | 0.25 = 0.0189 AU |
| Noise equivalent absorbance | −log (1 − 5.7e−9/4e−5) = |
| | 6 × 10⁻⁵ AU |

Technical Considerations

Signal to Noise ratio. Each red blood cell or erythrocyte is 6.5-8 microns in diameter. It can be important to deliver enough radiation to the cell to obtain a large enough signal to differentiate between the maternal and fetal SaO₂ state.

Current cell sort technologies can sort up to 10,000 cells per second. Spectroscopic determinations as described above can accommodate these rates.

Uses of the Separated Cells

Current technology can perform important diagnostic tests using cells separated by the methods described above. Tests developed in the future can also benefit from the cell separation capabilities of the present invention.

Current technology can not perform a standard karyotype using the fetal NRBC, since these cells cannot currently be cultured. Fetal cells that are obtained from invasive procedures generally allow for cell culture. These cells are mostly the fetal fibroblasts found in the amniotic fluid that grow well in culture media. This means that currently the cell sorting technology is limited to providing only genetic information on aneuploidy and a small limited number of other disease states. However, genetic aneuploidy comprises the majority of fetal DNA abnormalities.

There is currently a FISH probe that can be used to identify trisomy 13, 18, and 21 as well as to determine the sex of the cell. This is in commercial use and can be performed on cells that are unable to be cultured. This would allow for the above testing to be performed on fetal NRBC without the use of standard karyotyping. This would then allow for a “non-invasive” screening test to be employed that would account for approximately 80 percent of genetic anomalies as well as sex the fetus. The genetic field is a very rapidly expanding field. Although the current technology makes it difficult to obtain the complete genetic map from the fetal NRBC, future advances in testing might overcome this limitation.

Cell Lysis Example Application

The difference in response to radiation can also be used to selectively lyse cells. Consider the fetal/maternal blood cell properties discussed above as an example. Controlling the oxygen pressure environment allows production of a sample having oxygenated fetal cells and deoxygenated maternal cells. Due to the different oxygenation states, maternal cells absorb more energy at specific wavelengths. Irradiating the sample with radiation having a wavelength/intensity distribution sufficient to lyse the absorbing maternal cells but the not fetal cells can produce a sample with increased concentration of fetal blood cells. For example, radiation can be supplied at one absorbing wavelength, at an intensity such that maternal cells absorb a destructive level but fetal cells do not.

Those skilled in the art will recognize that the present invention may be manifested in a variety of forms other than the specific embodiments described and contemplated herein. Accordingly, departures in form and detail may be made without departing from the scope and spirit of the present invention as described in the appended claims.

We claim:

1. A method of determining the state of a cell containing a substance that exhibits different response to incident radiation depending on environmental conditions, comprising:
a. directing stimulus light to the cell, where the light comprises light at a signal wavelength that is characteristic of the different response;
b. detecting response light from the cell;
c. determining the state of the substance in the cell by comparing the stimulus light with the response light.

2. The method of claim 1, wherein the substance is hemoglobin, and the signal wavelength is selected to differentiate between hemoglobin types

3. The method of claim 2, wherein the signal wavelength is in the band illustrated in FIG. 1.

4. The method of claim 1, wherein the method is performed in vitro.

5. The method of claim 1, wherein the stimulus light comprises light at a plurality of wavelengths.

6. The method of claim 5, wherein determining the state of the substance comprises spectral analysis.

7. The method of claim 1, wherein the substance is hemoglobin.

8. The method of claim 1, wherein the cell is a red blood cell, and the substance is hemoglobin.

9. A method of determining whether a cell contains a specific type of hemoglobin, comprising;
a. establishing an environment surrounding the cell that corresponds with a known oxygenation state of the specific type of hemoglobin;
b. determining the oxygenation state of the cell by analyzing the cell's response to incident radiation;
c. determining whether the cell contains the specific type of hemoglobin by comparing the determined oxygenation state with the known oxygenation state.

10. A method of determining the environmental conditions surrounding a cell that contains a known type of hemoglobin, comprising:
a. determining the oxygenation state of the cell by analyzing the cell's response to incident radiation;
b. determining the environmental conditions surrounding the cell by comparing the determined oxygenation state with known relationship between oxygenation state and environmental conditions.

11. A method of classifying cells that are characterized by different hemoglobin content, comprising:
a. Establishing an environment surrounding the cells that causes different oxygenation states for cells having different hemoglobin content;
b. For each cell to be discriminated, determining the oxygenation state by analyzing the cell's response to incident radiation;
c. Classifying the cell by using said determined oxygenation state to identify cells with desired hemoglobin content.

12. A method of determining the relative proportions of different hemoglobin types in a blood sample, comprising;
a. controlling the environment surrounding the blood sample so that different oxygenation states occur with different hemoglobin types;
b. measuring the sample's response to incident light
c. using said measure light to select cells of a given hemoglobin type

13. A method of selectively altering one of two cell types in a sample, comprising directing incident light to cells in the sample, where the incident light has an intensity/wavelength characteristic that alters cells of the first type and does not alter cells of the second type.

14. A method of reducing the probability of the presence of a contaminant cell type from a sample of cells, comprising directing incident light to cells in the sample, where the incident light has an intensity/wavelength characteristic that disrupts cells of the contaminant cell type and does not disrupt cells not of the contaminant cell type.

15. A method of disrupting cells of a first type in a sample comprising cells of first and second types, where cells of the first type have an associated first light wavelength absorption characteristic, and where cells of the second type have an associated second light wavelength absorption characteristic, comprising directing incident light to cells in the sample, where the incident light has an intensity at a first disruptor wavelength sufficient to disrupt cells having the first light wavelength absorption characteristic and not sufficient to disrupt cells having the second light wavelength absorption characteristic.

16. The method of claim 15, wherein the first disruptor wavelength is a wavelength where cells of the first type are more strongly absorbing than cells of the second type.

17. A method of enriching the proportion of fetal blood cells in a sample containing fetal blood cells and maternal blood cells, comprising:
a. controlling the environment of the sample to produce oxygenated fetal blood cells and deoxygenated maternal blood cells;
b. directing light at a wavelength absorbed by deoxygenated cells more strongly than oxygenated cells with an intensity greater than disruption threshold for strong absorption and less than disruption threshold for weak absorption to cells in the sample.

18. A method of sorting, testing, or counting cells, comprising:
a. supplying cells to a single cell flow stream;
b. sorting, testing, or counting the cells based on the cells differential absorption of incident light.

19. The method of claim 18, wherein cells are supplied to a plurality of single cell flow streams, and further comprising combining the results of the sorting, testing, or counting of each of the plurality of single cell flow streams.

20. A method of removing cells of a first type from a sample, comprising:
a. supplying cells from the sample to a single cell flow stream;
b. identifying cells as they flow down stream as of the first type or not of the first type;
c. if a cell is identified as of the first type, then destroying it or identifying it for destruction;
d. collecting the cells as they exit the single cell flow stream.

21. The method of claim 20, wherein identification comprises identification using incident light at identification wavelength/intensity, and where destruction comprises supplying incident light having a wavelength/intensity characteristic sufficient to destroy the cell.

22. A method for the selection of cells containing fetal hemoglobin, comprising
a. exposing cells to a defined partial pressure of oxygen such that there is a difference in the absorbance characteristics of fetal hemoglobin and material hemoglobin;
b. using said difference in absorbance characteristic to identify the cells containing fetal hemoglobin; and
c. selecting the cells containing fetal hemoglobin.

23. The method of claim 22 further comprising:
a. examining the absorbance characteristics of the selected cells; and
b. identifying those cells with absorbance characteristics consistent with cells of fetal origin.

24. The method of claim 23 wherein determining absorbance characteristic consistent with cells of fetal origin comprises:
a. measuring the absorbance characteristic of each cell;
b. establishing an absorbance level that is consistent with cells of maternal origin; and
c. applying the established absorbance level to select those cells of fetal origin.

25. A method of identifying cells of a first type in a two-dimensional disposition of cells, comprising:
a. Controlling the environment affecting the cells such that there is differential oxygenation of cells of the first type relative to other cells in the disposition;
b. Determining an absorbance characteristic of the cells as related to the position in the two-dimensional disposition;
c. Determining from the absorbance characteristic the regions of the two-dimensional disposition containing cells of the first type.
